(19) United States
(12) Patent Application Publication
(10) Pub. No.: US 2025/0050135 A1
(43) Pub. Date: Feb. 13, 2025

(54) PATIENT POSITIONING APPARATUS FOR A RADIOTHERAPY SYSTEM

(71) Applicant: ELEKTA BEIJING MEDICAL SYSTEMS CO., LTD., Beijing (CN)

(72) Inventors: Fei YU, Beijing (CN); Guangrui GUO, Beijing (CN); Xueyuan ZOU, Beijing (CN)

(21) Appl. No.: 18/723,146

(22) PCT Filed: Dec. 21, 2021
(86) PCT No.: PCT/CN2021/139897

§ 371 Date: Jun. 21, 2024

(51) Int. Cl. A61N 5/10

(52) U.S. Cl. CPC A61N 5/1049; A61N 5/1075

(57) ABSTRACT

An approach for calibrating a radiotherapy positioning apparatus having a patient support surface that is moveable by a mechanism comprises obtaining, at multiple positions of the support surface, outputs of a patient support surface position sensor and a mechanism position encoder and using those outputs to determine a relationship between the sensor and encoder outputs.

This disclosure relates to apparatus, devices, systems, and approaches for radiotherapy, and in particular but without limitation to apparatus and/or systems for patient positioning.

### BACKGROUND

Radiotherapy can be described as the use of ionising radiation, such as X-rays, to treat a human or animal body. Radiotherapy is commonly used to treat tumours within the body of a human or animal patient, or subject. In such treatments, ionising radiation is used to irradiate, and thus destroy or damage, cells which form part of the tumour.

Precise control of patient position is important for effective radiotherapy. Complex patient positioning systems are used to move a patient to an intended position such that the patient can be appropriately irradiated by a treatment beam. In some systems, the patient may be translated and rotated with multiple dimensions of possible movement. In combination with treatment planning, and a moveable treatment beam, such an approach allows for the optimisation of the delivery of radiation to a tumour and can minimise the amount of healthy tissue that is exposed to radiation.

### SUMMARY

An invention is set out in the claims.

### FIGURES

Specific examples are now described, by way of example only, with reference to the drawings, in which:

FIG. 1 shows a radiotherapy system;

FIG. 2 shows a patient positioning apparatus in a first configuration;

FIG. 3 shows the patient positioning apparatus of FIG. 2 in a second configuration;

FIG. 4 shows a schematic of a patient support surface;

FIG. 5 shows a method for calibrating a patient positioning apparatus;

FIG. 6 shows a method for calibrating a patient positioning apparatus;

FIG. 7 shows a rotatable patient support surface; and

FIG. 8 shows a method for calibrating a patient positioning apparatus.

### DETAILED DESCRIPTION

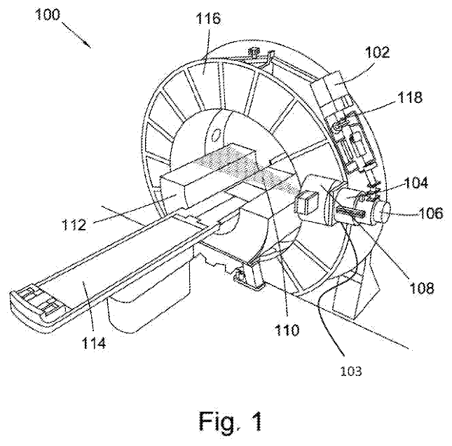

FIG. 1 shows a radiotherapy system, or device, suitable for delivering, and configured to deliver, a beam of radiation to a patient during radiotherapy treatment. The device and its constituent components will be described generally for the purpose of providing useful accompanying information for the present disclosure. The device shown in FIG. 1 is in accordance with the present disclosure and is suitable for use with the disclosed systems and apparatuses. While the device in FIG. 1 is an MR-linac (magnetic resonance linear accelerator), the implementations of the present disclosure may be any radiotherapy device, for example a linac (linear accelerator) device.

The device 100 shown in FIG. 1 is an MR-linac. The device 100 comprises both MR imaging apparatus 112 and radiotherapy (RT) apparatus which may comprise a linac device. The MR imaging apparatus 112 is shown in the diagram in a partially cut away perspective manner. In operation, the MR scanner produces MR images of the patient, and the linac device produces and shapes a beam of radiation and directs it towards a target region within a patient's body in accordance with a radiotherapy treatment plan. FIG. 1 does not show the usual ‘housing’ which would cover the MR imaging apparatus 112 and RT apparatus in a commercial setting such as a hospital.

The MR-linac device shown in FIG. 1 comprises a source 102 of radiofrequency (RF) waves, a waveguide 104, an electron source 106, a radiation source 103, a collimator 108 such as a multi-leaf collimator configured to collimate and shape the beam, MR imaging apparatus 112 (shown partially cut away), and a patient support surface 114. In use, the device would also comprise a housing (not shown) which, together with the ring-shaped gantry, defines a bore. The patient support surface 114 is moveable and can be used to support a patient and move them, or another subject, into the bore when an MR scan and/or when radiotherapy is to commence. The MR imaging apparatus 112, RT apparatus, and a patient support surface actuator are communicatively coupled to a controller or processor. The controller is also communicatively coupled to a memory device comprising computer-executable instructions which may be executed by the controller.

The RT apparatus comprises a radiation source 103 and a radiation detector (not shown). Typically, the radiation detector is positioned diametrically opposed to the radiation source 103. The radiation detector is suitable for, and configured to, produce radiation intensity data. In particular, the radiation detector is positioned and configured to detect the intensity of radiation which has passed through the subject. The radiation detector may also be described as radiation detecting means, and may form part of a portal imaging system.

The radiation source 103 may comprise a beam generation system. For a linac, the beam generation system may comprise a source 102 of RF waves, an electron source 106 such as an electron gun, and a waveguide 104. The radiation source 103 is attached to the rotatable gantry 116 so as to rotate with the gantry 116. In this way, the radiation source 103 is rotatable around the patient so that a treatment beam 110 can be applied from different angles around the gantry 116. In a preferred implementation, the gantry is continuously rotatable. In other words, the gantry can be rotated by 360 degrees around the patient, and in fact may continue to be rotated past 360 degrees. The gantry may be ring-shaped. In other words, the gantry may be a ring-gantry.

The source 102 of radiofrequency waves, such as a magnetron, is configured to produce radiofrequency waves. The source 102 of radiofrequency waves is coupled to the waveguide 104 via a circulator 118 and is configured to pulse radiofrequency waves into the waveguide 104. Radiofrequency waves may pass from the source 102 of radiofrequency waves through an RF input window and into an RF input connecting pipe or tube. The electron source 106 is also coupled to the waveguide 104 and is configured to inject electrons into the waveguide 104. In the electron source 106, electrons are thermionically emitted from a cathode filament as the filament is heated. The temperature of the filament controls the number of electrons injected. The injection of electrons into the waveguide 104 is synchronised with the pumping of the radiofrequency waves into the waveguide 104. The design and operation of the source 102 of radiofrequency waves, the electron source 106 and the waveguide 104 is such that the radiofrequency waves accelerate the electrons to very high energies as the electrons propagate through the waveguide 104.

The design of the waveguide 104 depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide 104. As the electrons are accelerated in the waveguide 104, the electron beam path is controlled by a suitable arrangement of steering magnets, or steering coils, which surround the waveguide 104. The arrangement of steering magnets may comprise, for example, two sets of quadrupole magnets.

Once the electrons have been accelerated, they may pass into a flight tube. The flight tube may be connected to the waveguide by a connecting tube. This connecting tube or connecting structure may be called a drift tube. The electrons travel toward a heavy metal target which may comprise, for example, tungsten. Whilst the electrons travel through the flight tube, an arrangement of focusing magnets act to direct and focus the beam on the target.

To ensure that propagation of the electrons is not impeded as the electron beam travels toward the target, the waveguide 104 is evacuated using a vacuum system comprising a vacuum pump or an arrangement of vacuum pumps. The pump system is capable of producing ultra-high vacuum (UHV) conditions in the waveguide 104 and in the flight tube. The vacuum system also ensures UHV conditions in the electron gun. Electrons can be accelerated to speeds approaching the speed of light in the evacuated waveguide 104.

The radiation source 103 is configured to direct the treatment beam 110 of therapeutic radiation toward a patient positioned on the patient support surface 114. The radiation source 103 may therefore also be referred to as a therapeutic radiation source. The radiation source 103 may comprise a heavy metal target towards which the high energy electrons exiting the waveguide are directed. When the electrons strike the target, X-rays are produced in a variety of directions. A primary collimator may block X-rays travelling in certain directions and pass only forward travelling X-rays to produce the treatment beam 110. The X-rays may be filtered and may pass through one or more ion chambers for dose measuring. The beam can be shaped in various ways by beam-shaping apparatus, for example by using the multi-leaf collimator 108, before it passes into the patient as part of radiotherapy treatment.

In some implementations, the radiation source 103 is configured to emit either an X-ray beam or an electron particle beam. Such implementations allow the device to provide electron beam therapy, i.e. a type of external beam therapy where electrons, rather than X-rays, are directed toward the target region as the therapeutic radiation. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The subject or patient support surface 114 is configured to move between a first position substantially outside the bore, and a second position substantially inside the bore. In the first position, a patient or subject can mount the patient support surface. The patient support surface 114, and patient, can then be moved inside the bore, to the second position, in order for the patient to be imaged by the MR imaging apparatus 112 and/or imaged or treated using the RT apparatus. The bore may hence lie about a portion of space that is suitable for receiving at least a portion of a patient-a patient receiving space. The movement of the patient support surface is effected and controlled by a patient support surface actuator, which may be described as an actuation mechanism. Together, these components may be described as a patient positioning system, or patient positioning apparatus, which may comprise other components. The actuation mechanism is configured to move the patient support surface in a direction parallel to, and defined by, the central axis of the bore. The terms subject and patient are used interchangeably herein such that the patient support surface can also be described as a subject support surface. The patient support surface may also be referred to as a moveable or adjustable couch or table.

The radiotherapy apparatus/device shown in FIG. 1 also comprises MR imaging apparatus 112. The MR imaging apparatus 112 is configured to obtain images of a subject positioned, i.e. located, on the patient support surface 114. The MR imaging apparatus 112 may also be referred to as the MR imager. The MR imaging apparatus 112 may be a conventional MR imaging apparatus operating in a known manner to obtain MR data, for example MR images. The skilled person will appreciate that such an MR imaging apparatus 112 may comprise a primary magnet, one or more gradient coils, one or more receive coils, and an RF pulse applicator. The operation of the MR imaging apparatus is controlled by the controller.

The controller is a computer, processor, or other processing apparatus. The controller may be formed by several discrete processors; for example, the controller may comprise an MR imaging apparatus processor, which controls the MR imaging apparatus 110; an RT apparatus processor, which controls the operation of the RT apparatus; and a subject support surface processor which controls the operation and actuation of the patient support surface. The controller is communicatively coupled to a memory, e.g. a computer readable medium.

The linac device also comprises several other components and systems as will be understood by the skilled person. For example, in order to ensure the linac does not leak radiation, appropriate shielding is also provided.

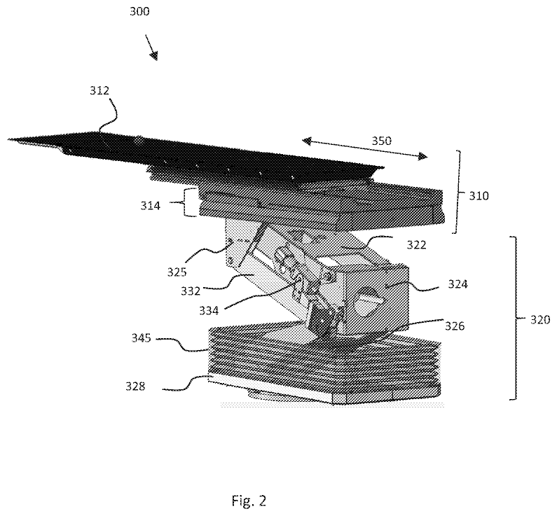

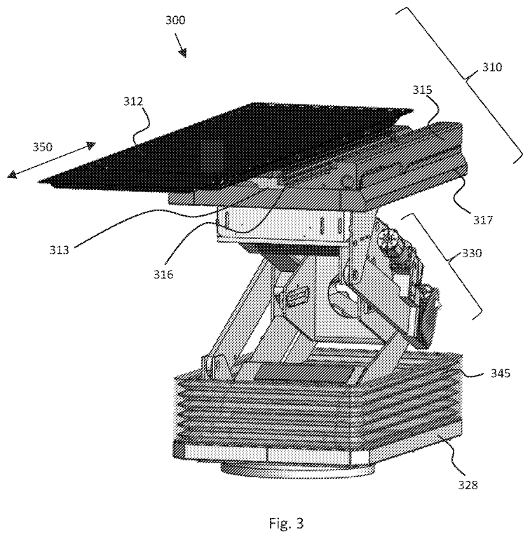

FIG. 2 and FIG. 3 show a patient positioning apparatus 300. FIG. 2 shows an angled rear view of the patient positioning apparatus 300. FIG. 3 shows an angled front view of the positioning apparatus 300. The patient positioning apparatus 300 comprises a patient support apparatus 310 and a support structure 320. The support structure 320 is configured to support the patient support apparatus 310 above a floor, such as the floor of a treatment room. The support structure 320 may be configured to provide this support, in part, by means of a base 328 which contacts the floor, and/or which is embeddable within the floor. In the implementation depicted in FIGS. 2 and 3, the support structure comprises a first, or upper, supporting leg 322, a support element 324, and a second, or lower, supporting leg 326.

The patient positioning apparatus 300 also comprises a rotation mechanism. The rotation mechanism is configured to tilt, i.e. rotate, the patient support apparatus 310. The rotation is made with respect to a horizontal plane, or equivalently with respect to the floor of the treatment room, in order to adjust a tilt angle, for example a pitch angle. In the implementation shown in FIGS. 2 and 3, the rotation mechanism comprises a drive member 332 and an actuation mechanism 330. The rotation mechanism may also comprise a coupling element 325 which couples the drive member 332 to the patient support apparatus 310. The rotation mechanism is configured to impart a force, via the drive member 332, to an underside of the patient support apparatus 310 to thereby rotate the patient support apparatus 310 with respect to the support structure 320.

The patient support apparatus 310 is configured to support a patient. The patient support apparatus 310 comprises a patient support surface 312 and a patient support base 314. In use of the apparatus, a patient may lie on the patient support surface 312. In other words, in use, the patient contacts an upper surface of the patient support apparatus 310. The patient support surface 312 is coupled to the patient support base 314 using a linear guide. As will be known to those skilled in the art, a linear guide is arranged to hold two components relatively rigidly with respect to each other via a coupling mechanism that allows translational motion of the components relative to each other back and forth along a particular axis but will prevent lateral and/or twisting motion of the two components relative to one another. The patient support surface 312 can be moved linearly with respect to the patient support base 314 along a direction parallel with the longitudinal axis of the patient support apparatus 310. The directions of this linear movement are indicated by the double-headed arrow 350. Movement of the patient support surface 312 with respect to the patient support base 314 is controlled via a linear actuator.

As can be seen in FIG. 3, a supporting structure 315 of the patient support base 314 is coupled to an intermediate support structure 313 of the patient support surface 312 via the linear guide. As will be appreciated by those skilled in the art, the coupling shown in FIGS. 2 and 3 may be considered as a single linear guide or as two linear guides. The linear guide comprises an elongate bearing guide 316 that is mounted to the intermediate support structure 313. In some examples, an elongate bearing guide is instead mounted to the supporting structure 315 and in other examples, an elongate bearing guide is mounted to each of the intermediate support structure 313 and the supporting structure 315. Likewise, more than one pair of elongate bearing guides may form the linear guide. At least one bearing is provided to move relative to the elongate bearing guide, enabling the linear movement. The bearing may be mounted or attached to one of the supporting structure 315 or the intermediate support structure 313, or may not be mounted or attached to either the supporting structure 315 or the intermediate support structure 313—as may be the case when the system is being assembled, disassembled, or tested. Whichever arrangement is used, the respective components allow the patient support surface 312 to be coupled to and linearly translated relative to the patient support base 314.

Separate to the linear guide described above, the patient positioning apparatus 300 may be configured to rotate the patient support surface 312 with respect to one or both of a pitch and a roll rotation axis. In such an implementation, the axis of linear movement of the patient support surface 312 with respect to the patient support base 314 may be parallel with the roll rotation axis. In alternative implementations, the axis of linear movement of the patient support surface 312 with respect to the patient support base 314 and the roll rotation axis may not be parallel. The roll rotation movement and/or the pitch rotation movement may be controlled via a linear actuator or suitable actuation mechanism 330.

In addition, or as an alternative, to a longitudinal movement, the patient positioning surface may also be configured to move laterally. This movement may be perpendicular to the longitudinal movement and can be controlled via movement of the supporting structure 315 with respect to a lateral sledge 317. This movement can be effected by actuators in a known way, and may also make use of one or more linear guides. In summary, the patient support surface 312 may be configured to move in any, all, or a combination of three translator degrees of freedom: height, a longitudinal movement and a lateral movement.

The support structure 320 is configured to bear the weight of the patient support apparatus 310, as well as a patient positioned on the patient support surface 312. Multiple implementations of the support structure 320 are envisaged. In the implementation depicted in FIGS. 2 and 3, the support structure 320 comprises an upper element coupled to an underside of the patient support apparatus 310, and a lower element coupled to the base. The upper element may be a first supporting leg 322 and the lower element may be a second supporting leg 326 coupled to the base. The first supporting leg 322 and second supporting leg 326 are coupled to one another via a support element 324. The support element 324 may be referred to as a support block or anchor element herein.

The patient support apparatus 310 is rotationally coupled to the support structure 320 to allow rotation about a rotation axis. In a simple implementation, the support structure 320 may be coupled to the patient support apparatus 310 via an interface between a shaft and one or more bearings which receive the shaft. For example, the one or more bearings may be mounted to an underside of the patient support apparatus 310, and configured to receive a shaft which forms part of the support surface. For example, an upper region of the first supporting leg 322 may culminate in a double-ended shaft, with each end of the shaft being received in a bearing mounted to a base of the patient support apparatus 310. In this implementation, the orientation of the shaft and bearings defines an axis of rotation about which the patient support apparatus 310 may rotate with respect to the support structure 320. Other implementations include a ball-joint, or any other mechanical connection that allow rotation of the patient support apparatus 310 with respect to the support surface via a rotation axis.

A second, or lower, supporting leg is coupled to the base 328. The second supporting leg 326 may be fixedly attached to the base 328. Alternatively, the coupling may be achieved via a lower coupling element and the second supporting leg 326 may be configured to rotate with respect to the lower coupling element as part of a height adjustment mechanism. The lower coupling element extends upward away from the base 328, allowing the second supporting leg 326 to be coupled to the lower coupling element. Such an arrangement defines a rotation axis parallel with the rotation axis about which the patient support apparatus 310 rotates with respect to the first supporting leg 322.

The support structure 320 may also comprise a height adjustment mechanism (not shown in the figures). The height adjustment mechanism is configured to adjust the height, i.e. vertical distance, of the patient support apparatus 310 above the floor or base. The height adjustment mechanism comprises one or more motor mechanisms. An upper motor mechanism may be positioned within, form part of, and/or be coupled to, the support element 324. A lower motor mechanism may be positioned within, form part of, and/or be coupled to, the lower coupling element.

The height adjustment mechanism may be formed by one or multiple different mechanisms. In the implementation shown in FIG. 2 and FIG. 3, the height adjustment mechanism is configured to adjust the vertical distance between the support element 324 and the patient support apparatus 310 by actuating rotation of the first supporting leg 322 relative to the base 328. Thereby, the height of the patient support apparatus 310 above the floor is increased. The height adjustment mechanism comprises a rotational mechanism or motor configured to produce a rotary motion of the first supporting leg 322 with respect to the support element 324. This may be a rotary hydraulic motor. This rotary motor is housed within the support element 324. It will be appreciated that by rotating the first supporting leg 322 clockwise from the perspective shown in FIG. 2, the height of the patient support apparatus 310 above the floor/base is increased.

Optionally, an additional rotary motor may be provided. This rotary motor may be referred to as a ‘lower’ rotary motor in contrast to the ‘upper’ rotary motor described above. The lower rotary motor is also housed within the support element 324 and is configured to drive rotation with respect to the support element 324 and the lower leg 326. The height adjustment mechanism may thereby also be configured to adjust the vertical distance between the support element 324 and the base 328 and/or floor of the treatment room, by actuating the second supporting leg 326 using this lower rotary motor. Thereby, the height of the patient support apparatus 310 is adjusted. By synchronously driving rotation using both the upper and the lower rotary motor, the vertical height of the patient support surface 312 may be adjusted.

For example, the height adjustment mechanism may comprise a lower rotational mechanism or motor, e.g. a rotary hydraulic motor, configured to produce a rotary motion of the second supporting leg 326 with respect to the support structure 324. It will be appreciated that by rotating the second supporting leg 326 anti-clockwise, from the perspective shown in FIG. 2, the height of the patient support apparatus 310 above the base is increased.

The height adjustment mechanism is configured to control a height of the patient support apparatus 310 above the floor of the treatment room. As described above, the patient support apparatus 310, and in particular the base of the patient support apparatus 310, is rotationally coupled to the support structure 320 to allow rotation about a rotation axis. By adjusting the height of the patient support apparatus 310 above the floor of the treatment room using the height adjustment mechanism, the height of this rotation axis can also be adjusted.

Described herein is a support structure 320 and height adjustment mechanism which comprises a mechanism capable of rotating one or a plurality of supporting legs about rotation axes in order to adjust the height of the patient support apparatus 310. However, the height adjustment mechanism may take multiple forms. For example, the height adjustment mechanism may comprise an arrangement of hydraulic pistons positioned and configured to adjust the height of the patient support apparatus 310. An alternative implementation may involve a scissor lift mechanism. The skilled person will be aware of other possible ways in which the height of a patient support apparatus 310 may be adjusted. Regardless of the specific implementation of the support structure 320 and/or height adjustment mechanism, the rotation mechanism is coupled to the support structure 320 and is configured to impart a force to an underside of the patient support apparatus 310 in order to rotate the patient support apparatus 310 with respect to the support structure 320.

In some implementations, the positioning apparatus 300 also comprises a skirt 345 (configured to cover the support structure 320 and rotation mechanism). The skirt 345 is connectable between the base 328 and the patient support apparatus 310. The skirt 345 has a flexibility, and in particular may have a concertina configuration, i.e. be configured to extend, compress, or collapse in folds like those of a concertina. Thus, patients and clinicians are protected from possible injury due to the moving mechanisms described herein. It is simpler to provide this protection using a simple skirt 345 by virtue of the present design, and in particular by virtue of the rotation mechanism being attached to and supported by the support structure. In FIG. 2 and FIG. 3, the skirt 345 is shown as folded or compressed down away from the patient support apparatus 310 so that the support structure 320 and rotation mechanism can be seen.

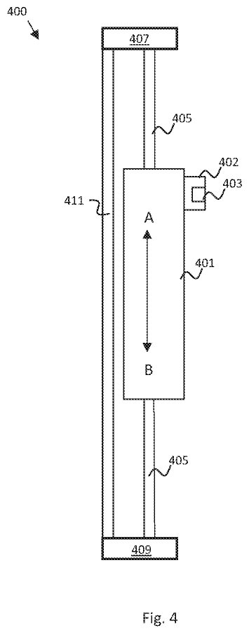

FIG. 4 is a schematic of an arrangement 400 for linear translation of a patient support surface 401. The patient support surface is arranged to be moved by a movement mechanism 402. The patient support surface 401 may be coupled to the movement mechanism 402 via intermediate components (not shown). An encoder 403 is arranged to record and/or output an internal position of the movement mechanism 402. The patient support surface 401 is arranged such that the movement mechanism 402 can move the patient support surface 401 along a track 405 in either of the directions indicated by the double-headed arrow running from A-B in FIG. 4. At either end of the track 405 is an end stop 407, 409 which prevents the patient support surface 401 from moving any further in a particular direction and sets a limit to the motion of the patient support surface 401 in that particular direction. Each end stop 407, 409 may be a mechanical block that is arranged such that the patient support surface 401 will abut it. A sensor 411 is arranged to determine the position of the patient support surface 401 along the track 405. The sensor 411 may be an absolutely linear sensor, such as a linear magnetic encoder available from Renishaw, MicroE, or Heidenhain. The sensor 411 may also be referred to as a magnetic ruler. The sensor 411 thus gives a direct indication of the position of the patient support surface 401.

The patient support surface 401 may be similar or identical to the patient support surface 312 of FIGS. 2 and 3. FIG. 4 may be considered a simplified depiction of the patient positioning apparatus 300. The movement mechanism 402 may likewise be any of the translation mechanisms disclosed herein, such as a linear or rotary actuator. For example, the movement mechanism 402 may comprise a mechanism for mechanically converting motion of an actuator component to motion of the patient support surface 401. The movement mechanism 402 may comprise a motor with a gear box, and/or a transmission device, and/or a solenoid. The track 405 and the directions A-B may run along either of the longitudinal and lateral directions described herein, or along the height direction Z.

The encoder 403 may record the position of a component of the movement mechanism 402 such as the position of a rotary component of a linear actuator or motor, or the number of rotations of the rotary component in a particular direction. The component of the movement mechanism 402 may hence be referred to as an internal component of the movement mechanism 402. The encoder may be a rotary encoder such as an optical rotary encoder, such as those available from Posital, Hengstler, or Heidenhain. In an ideal system, as the movement mechanism 402 moves the patient support surface 401, the change in position or orientation of the internal components of the mechanism corresponds exactly to the change in position of the patient support surface 401, and the reading of the encoder 403 may give a direct indication of the position of the patient support surface 401. However, typically the internal component(s) of the movement mechanism will 402 exhibit characteristics such as backlash, and the change in position or orientation of the internal component will not correspond directly to a change in position of the patient support surface 401. Thus the encoder may not always give a direct indication of the position of the patient support surface 401.

The absolutely linear output or reading of the sensor 411 is typically a true indication of the position of the patient support surface 401, in contrast to the reading of the encoder 403. The output of the sensor 411 can thus be considered to provide an absolute measurement of the position of the patient support surface 401 along the track 405, whereas the output of the encoder 403 can be considered to provide an approximate or relative measurement of the change of position of the patient support surface 401.

The sensor 411 measures the absolute position of the patient support surface 401 with a particular spatial resolution. As the patient support surface 401 is driven along the track 405 by the movement mechanism 402, the sensor 411 is sensitive only to changes in position that exceed the resolution of the sensor 411. Similarly, as the movement mechanism 402 drives the patient support surface 401 along the track, the encoder 403 records the changing position of the internal component of the movement mechanism 402 with a particular resolution. Typically, the smallest change of the internal position that the encoder can resolve would cause a positional change in the patient support surface that is smaller than a smallest change in position of the patient support surface that the sensor 411 can resolve. The sensor 411 may thus be considered to be of lower resolution than the encoder 403 with respect to measuring the position of the patient support surface 401.

The sensor 411 and the encoder 403 thus each provide different advantages and disadvantages. Disclosed herein is a system and/or method for combining the sensor 411 and the encoder 403 readings in a way that mitigates the disadvantages of each of the sensor and encoder and promotes the advantages of each of the sensor and encoder.

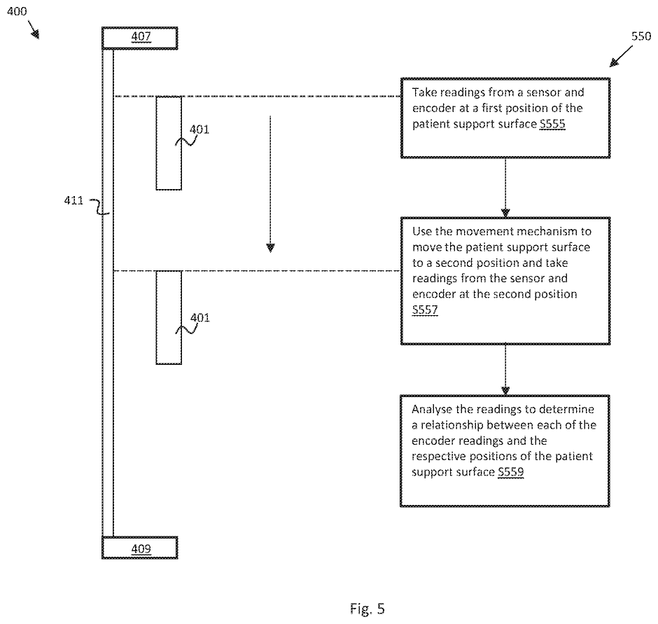

FIG. 5 shows a further-simplified schematic of the arrangement 400 of FIG. 4 alongside a method 550 for calibrating a patient positioning apparatus. For simplicity, the movement mechanism 402, encoder 403, and track 405 are not shown.

At a first step S555, the method 550 comprises taking readings from the sensor 411 and the encoder 403 at a first position of the patient support surface. The absolute position of the patient support surface 401 may be determined from the reading of the sensor 411. The relative position of the patient support surface according to the encoder 403 reading may thus be converted into an absolute position based on the reading of the sensor 411.

At a second step S557, the method 550 comprises using the movement mechanism 402 to move the patient support surface 401 to a second position and taking readings from the sensor 411 and the encoder 403 at the second position. The absolute position of the patient support surface 401 at the second position may be estimated or determined based on the reading of the sensor 411. The relative position of the patient support surface at the second position according to the encoder 403 reading may thus be converted into an absolute position based on the reading of the sensor 411.

At a third step S559, the method 550 comprises analysing the readings to determine a relationship between each of the encoder readings and the respective positions of the patient support surface. Since the encoder reading or output is known for two positions and the absolute position is also known for each of those two positions, a suitable interpolation, extrapolation, or determination may be made such that encoder readings at other positions may be converted to an absolute position measurement or estimate.

Beneficially, the determined relationship between each of the encoder readings and the respective positions of the patient support surface allows the encoder reading to be used as a direct measurement or estimate of the position of the patient support surface. Thus, the higher resolution of the encoder 403 may be utilised to provide more precise estimates of position than those of the sensor 411. Furthermore, the reliability of the absolutely linear sensor 411 may be used to guarantee the integrity of the position determined from the encoder 403.

In the methods disclosed herein, the output of the sensor 411 may be related to the position of the patient support surface 401 according to the equation:

$$P = {cE}^{′} + d \qquad ( 1 )$$

Where P is the true position of the patient support surface 401, E′ is the output or reading of the sensor 411, and c is determined by the sensor 411 manufacturing process and typically has very high accuracy. In many implementations, c is effectively equal to 1. d is an offset caused by the mounting and/or cut position of the sensor 411.

In the methods disclosed herein, the output of the encoder 403 may be related to the position of the patient support surface 401 according to the equation:

$$P = aE + b \qquad ( 2 )$$

Where P is the true position of the patient support surface 401, E is the output or reading of the encoder 403, a is determined by at least one characteristic of the movement mechanism 402 or patient positioning apparatus, and b is an offset caused during assembly. The parameter a may represent a characteristic of one or more internal components of the movement mechanism 402, such as of a transmission device and/or a gearbox. In some examples, the parameter a may represent the length of a guide, an angle of a component part, and/or the position of a screw. In each example, the parameter a may have a theoretical reference value according to the specification of the represented component(s). The value of the parameter a may vary from the theoretical reference value due to the manufacturing and/or installation process.

Determining a relationship between each of the encoder readings and the respective positions, as in the method 550 of FIG. 5, may comprise solving the simultaneous equations (1) and (2) using the readings taken at steps S555 and S557.

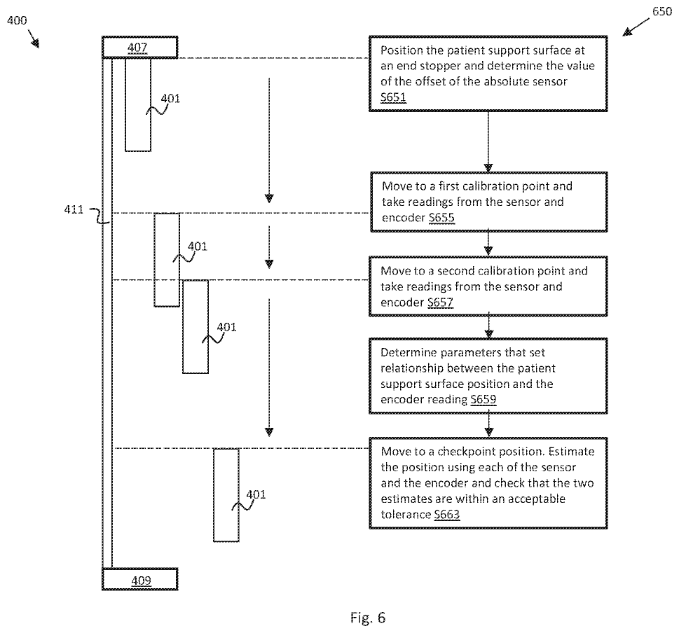

FIG. 6 shows the further-simplified schematic of the arrangement 400 of FIG. 5 alongside a method 650 for calibrating a patient positioning apparatus. The method 650 is an exemplary implementation of the method 550 of FIG. 5 with the addition of optional further steps.

At a first step S651, the patient support surface 401 is positioned at one end of the track 405, against an end stop 407, 409. The end of the track 405 can be considered to be a zero position, i.e. P=0. Assuming that c is equal to 1, the value of the offset parameter d can thus be calculated at P=0. The value of d is constant across the range of motion of the patient support surface, meaning that P can be determined for any value of E, providing a measurement of the true position of the patient support surface 401 along the track 405. In some examples, the offset value d may already be known and the first step S651 may not be required.

At a second step S655, the patient support surface 401 is moved, using the movement mechanism 402, to a first position that is different to the end or zero position of the first step S651. The first position is a first calibration position. At the first position, readings are taken from the sensor 411 and the encoder 403, as per the first step S555 of FIG. 5. The readings provide values for E and E′ at the first position.

At a third step S657, the patient support surface 401 is moved, using the movement mechanism 402, to a second position that is different to the first position. The second position is a second calibration position. At the second position, readings are taken from the sensor 411 and the encoder 403, as per the second step S557 of FIG. 5. The readings provide values for E and E′ at the second position.

At a fourth step S659, the parameters that define a relationship between the patient support surface position and the encoder reading are determined. The simultaneous equations (1) and (2) are solved using the readings for each of the two calibration positions and a and b are determined. Optionally, the determined value of a is compared with a theoretical reference value. The theoretical reference value may be stored or preset in a computer memory, such as in a look-up table. Optionally, if the value of a does not match the theoretical reference value, the method may comprise raising, producing, or activating an alarm indicating that calibration has not succeeded. The method may further comprise restarting the method 650 if the value of a does not match the theoretical reference value.

At a fifth step S663, the method comprises using the movement mechanism 402 to move the patient support surface 401 to a third position that is different to the first position and the second position. The third position is a checkpoint position. Readings are taken from the sensor 411 and the encoder 403 and equations (1) and (2), along with the previously determined values for a, b, c and d, are used to convert each respective reading into a respective estimate or calculation of the position of the patient support surface 401. The fifth step S663 may also be performed as part of the method 550 of FIG. 5, wherein two estimates may be made of the checkpoint position based on the respective readings of the sensor 411 and the encoder 403 in combination with the determined relationship.

Hence, two independently-sourced estimates of the position are obtained at the checkpoint position. The estimate obtained using equation (1) is likely to be the true position of the patient support surface 401. If the calibration method has worked and a and b have been determined with sufficient accuracy, then the estimate obtained using equation (2) is also likely to be the true position of the patient support surface 401. The two estimates of position are compared at the fifth step S663, such as by determining the difference between the two values or by taking a ratio of the two values. If the difference or ratio is not as expected, for example if there is a large difference between the two estimates, it may indicate that an problem has occurred. Optionally, a determination is made as to whether the comparison between the two estimates is within a predefined tolerance. If the comparison determines that the two estimates are outside of the predefined tolerance, an alarm may be raised or activated and/or the calibration method may be restarted. Possible problems include: an unexpected change or failure in the movement mechanism 402, that the prior calibration steps of the method were not performed correctly or accurately, and that either of the sensor 411 or the encoder 403 has failed in some way.

If the comparison determines that the two estimates are within the predefined tolerance, the patient support apparatus is considered to be correctly calibrated. Moreover, improved accuracy and control of the patient support apparatus can be achieved by using the higher resolution encoder 403 to determine the absolute position of the patient support surface 401, rather than using the lower resolution sensor 411. Moreover, independent estimation and verification by the comparison of the fifth step S663 may be performed each time the patient support surface 401 is moved to a new position. Such an approach allows the calibration of the patient positioning apparatus to be continuously verified during use. Beneficially, an approach is provided wherein if a positioning error occurs during use, such as when positioning a patient, then an alarm or indication is raised immediately.

Each of the X, Y, and Z axes of motion of the patient positioning apparatus may be calibrated thus and movement of the patient support surface may be continuously monitored and/or verified to ensure that the patient positioning apparatus remains correctly calibrated in each direction of motion.

Rotational motion of the patient positioning apparatus may also be calibrated according to the methods disclosed herein.

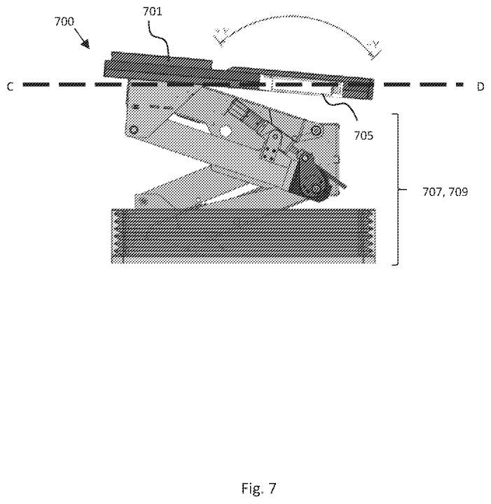

FIG. 7 shows an arrangement 700 comprising a rotatable patient support surface 701. The patient support surface 701 may be equivalent to other patient support surfaces 401, 312 disclosed herein and may be part of any of the patient positioning apparatus disclosed herein. Indeed, the arrangements 400, 700 of FIGS. 4 and 7 may be the same apparatus. Each figure is merely a simplified depiction used to illustrate a dimension of possible motion of the patient support surface.

The patient support surface 701 is rotatable in the directions indicated by the double-headed arrow running between +Y and −Y. Such rotation is called pitch rotation. Although pitch motion is discussed herein for illustrative purposes, the patient support surface 701 may also be rotatable around other axes perpendicular to that of the pitch rotation, such as roll and yaw rotation, and the methods and systems disclosed herein apply likewise to those rotation directions.

The degree of rotation of the patient support surface 701 is measured as an angle relative to the dashed line C-D, which runs parallel to the floor on which the patient positioning apparatus is situated. The degree of rotation, or tilt, or inclination, of the patient support surface 701 is measured using a sensor 705. The sensor 705 is an absolute sensor like the sensor 411 of FIGS. 4 to 6. The sensor 705 may be an inclinometer and/or arranged to measure the absolute rotational position of the patient support surface with respect to the direction of gravity towards earth. A movement mechanism 707 is arranged to cause rotation of the patient support surface 701. The movement mechanism 707 may be any of those disclosed herein, and may include components such as gears, linear or rotary actuators, and/or transmission drives. The movement mechanism 707 comprises an encoder 709 (not shown—in this example it is internal to the movement mechanism 707) that is similar to the encoder 403 of FIGS. 4 to 6 and is arranged to measure an internal position of the movement mechanism 707.

Calibrating rotational motion of the patient support surface can be considered in an analogous manner to the calibration approach disclosed for linear translation in FIGS. 4 to 6. Instead of measuring positions along a linear track, different angular/rotational positions or orientations of the patient support surface are measured. The sensor 705 of FIG. 7 provides an absolute measurement of position in a similar manner to that of the sensor 411 of FIG. 4 and the encoder 709 of FIG. 7 provides an indirect, relative measurement of position in a similar manner to that of the encoder 403 of FIG. 4. Likewise, the encoder 709 is typically of higher equivalent resolution than the sensor 705.

The sensor 705 provides an absolute measurement that has no offset. Accordingly, the rotational position of the patient support surface 701 is given by:

$$P = {E}^{′} \qquad ( 3 )$$

Where E′ is the output or reading of the sensor 705.

Due to the complexity of rotational motion mechanisms, the relationship between the output of the encoder 709 and the rotational position of the patient support surface 701 is dependent upon a function f(E) as follows:

$$P = f ( E ) \qquad ( 4 )$$

f(E) has several parameters, of number n, to be calibrated, each of which may relate to a characteristic of the movement mechanism 707 or the patient positioning apparatus. Each of the n parameters has a theoretical reference value. Typically, f(E) comprises multiple trigonometric functions of number n, each trigonometric function having a parameter, the parameter being one of the n parameters to be calibrated. The multiple trigonometric functions of f(E) are combined in a way that best describes the type of rotational motion to be characterised and/or calibrated. For example, when calibrating pitch rotation of the patient support surface 701, 9 parameters may be used, and/or when calibrating roll rotation of the patient support surface 701, 10 parameters may be used.

Equations (3) and (4) can be considered as analogous to the equations (1) and (2) that describe linear translational motion.

It will be appreciated that the exact form(s) of f(E) may be chosen such that it best describes or characterises the corresponding type of rotation of the patient support surface 701. In a manner analogous to that of the parameter a of equation (2), each of the n parameters of the function f(E) may represent one or more of the design characteristics of the movement mechanism 707, such as the length of a guide, an angle of a component part, and/or the position of a screw. Each parameter may vary from a theoretical reference value due to the manufacturing and/or installation process. A mathematical optimisation algorithm may be used to estimate the real value of the parameters in order to best fit f(E) according to the equation (4).

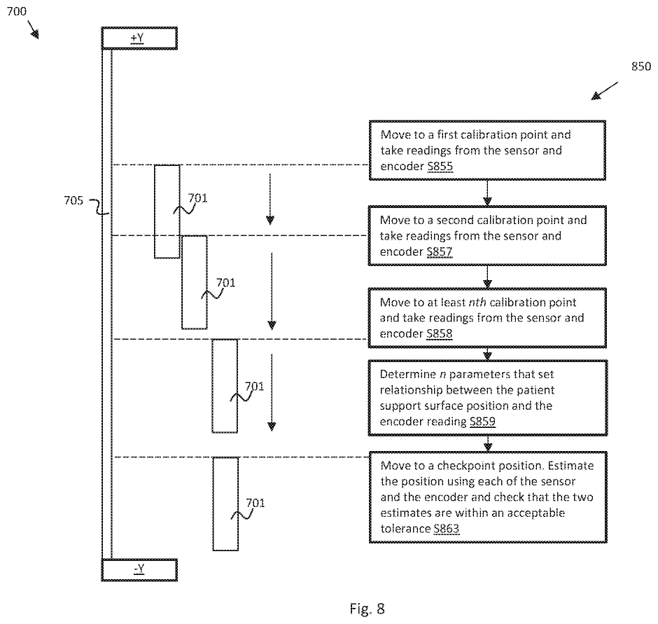

FIG. 8 shows a simplified schematic of the arrangement 700 of FIG. 7 alongside a method 850. The sensor 705 is illustrated as a linear scale spanning the maximum range of rotation of the patient support surface 701, +Y to −Y. In some examples, +Y is +3 degrees and −Y is −3 degrees. In other examples, different limits to the range of rotation will be set for a particular patient positioning apparatus.

At a first step S855, the method comprises rotating the patient support surface 701, using the movement mechanism 707, to a first rotational position or orientation. The first position is a first calibration position. At the first position, readings are taken from the sensor 705 and the encoder 709, as per the first step S555 of FIG. 5. The readings provide values for E and E′ at the first position.

At a second step S857, the patient support surface 701 is rotated to a second rotational position that is different to the first position. The second position is a second calibration position. At the second position, readings are taken from the sensor 705 and the encoder 709, as per the second step S557 of FIG. 5. Those readings provide values for E and E′ at the second position.

Further similar steps to the prior two steps S855, S857 should be performed such that the number of different positions at which readings are taken corresponds to the number of parameters n that must be calibrated for f(E) in equation (4). FIG. 8 shows a third step S858 representing the last such calibration step.

At a subsequent t step S859, the parameters that determine a relationship between the patient support surface position and the encoder reading are determined. Having taken readings from the sensor 705 and encoder at n positions, the n parameters of f(E) can be determined by solving equations (3) and (4) as simultaneous equations. In each case, the sensor 705 provides the true angular position or orientation of the patient support surface 701.

As with the method 650 of FIG. 6, each of the n parameters may be compared to a theoretical reference value. If the parameter does not match the theoretical reference value, an alarm may be raised or activated indicating that calibration has failed and/or the calibration process may be restarted. The theoretical reference value may be stored or preset in a computer memory, such as in a look-up table.

At a final step S863, the method comprises using the movement mechanism 707 to move the patient support surface 701 to a checkpoint position. Readings are taken from the sensor 705 and the encoder 709 and equations (3) and (4), along with the previously determined values for the n parameters, are used to convert each respective reading into a respective estimate or calculation of the rotational position of the patient support surface 701.

As described above in relation to linear translational motion, two independent estimates of the position are obtained at this step. The estimate obtained using equation (3) is likely to be the true rotational position of the patient support surface 701. If the calibration method has worked and the n parameters have been determined with sufficient accuracy, then the estimate obtained using equation (4) is also likely to be the true position of the patient support surface 701. The two estimates of position are compared at the final step S863, such as by determining the difference between the two values or by taking a ratio of the two values. If the difference or ratio is not as expected, for example if there is a large difference between the two estimates, it may indicate that an problem has occurred. Optionally, a determination is made as to whether the comparison between the two estimates is within a predefined tolerance. If the comparison determines that the two estimates are outside of the predefined tolerance, an alarm may be raised or activated and/or the calibration method may be restarted. Possible errors include: failure of the movement mechanism, that the prior calibration steps of the method were not performed correctly or accurately, and that either of the sensor 705 or the encoder 709 has failed in some way.

If the comparison determines that the two estimates are within the predefined tolerance, the patient support apparatus is considered to be correctly calibrated. Moreover, improved accuracy and control of the patient support apparatus can be achieved by using the higher resolution encoder 709 to determine the absolute position of the patient support surface 701, rather than using the lower resolution sensor 705. Moreover, independent estimation and verification by the comparison of the final step S863 may be performed each time the patient support surface 701 is moved to a new position. Such an approach allows the calibration of the patient positioning apparatus to be continuously verified during use. Beneficially, an approach is provided wherein if a positioning error occurs during use, such as when positioning a patient, then an alarm or indication is raised immediately.

Although each of the methods 550, 650, 850 is described herein in relation to a particular example, each method may be combined with another method or applied to another example. For example, there may be situations in which the method 850 of FIG. 8 may be applied to linear translation motion or the method 550 of FIG. 5 may be applied to rotational motion, so long as the relationship between the encoder output and the position of the patient support surface is appropriately determined.

There is described herein an approach for calibrating a radiotherapy positioning apparatus having a patient support surface that is moveable by a mechanism that comprises obtaining, at multiple positions of the support surface, outputs of a patient support surface position sensor and a mechanism position encoder and using those outputs to determine a relationship between the sensor and encoder outputs.

Those skilled in the art will recognise that a wide variety of modifications, alterations, and combinations can be made with respect to the above described examples without departing from the scope of the disclosed concepts, and that such modifications, alterations, and combinations are to be viewed as being within the ambit of the disclosed concepts.

Those skilled in the art will also recognise that the scope of the invention is not limited by the examples described herein, but is instead defined by the appended claims.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer-readable media or, more generally, a computer program product. The computer-readable media may be transitory or non-transitory. The one or more computer-readable media could be, for electronic, example, an magnetic, optical, electromagnetic, infrared, or semiconductor system, or a propagation medium for data transmission, for example for downloading the code over the Internet. Alternatively, the one or more computer-readable media could take the form of one or more physical computer-readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

There is described a computer-readable medium comprising computer-readable instructions which, when executed by a processor, cause the processor to perform any of the methods described herein. The computer-readable medium may be a tangible and/or non-transitory medium.

1. A method for calibrating a radiotherapy patient positioning apparatus, the radiotherapy patient positioning apparatus comprising:
a patient support surface;
a sensor for measuring a position of the patient support surface;
a movement mechanism for moving the patient support surface; and
an encoder for encoding an internal position of the movement mechanism, wherein a smallest change of the internal position that the encoder is able to resolve causes a positional change in the patient support surface that is smaller than the smallest change in position of the patient support surface that the sensor is able to resolve, the method comprising:
taking one or more readings from the sensor and encoder at a first position of the patient support surface;
using the movement mechanism to move the patient support surface to a second position and taking one or more readings from the sensor and encoder at the second position; and
analyzing the one or more readings from the sensor and encoder at the first position of the patient support surface and the one or more readings from the sensor and encoder at the second position of the patient support surface to determine a relationship between each of the encoder readings and the respective positions of the patient support surface.

2. The method of claim 1, wherein the method further comprises:
using the movement mechanism to move the patient support surface to a third position;
making a first estimate of the third position, the first estimate based on at least one of the one or more readings from the sensor;
using the determined relationship to make a second estimate of the third position, the second estimate based on at least one of the one or more readings from the encoder; and
comparing the first estimate and the second estimate of the third position.

3. The method of claim 2, wherein comparing the first estimate and the second estimate comprises determining whether the first estimate is within a predefined tolerance of the second estimate.

4. The method of claim 3, further comprising:
activating an alarm when the first estimate is not within the predefined tolerance of the second estimate.

5. The method of claim 1, wherein the sensor is arranged to measure an absolute position of the patient support surface along a linear track.

6. The method of claim 1, wherein the sensor is arranged to measure an absolute rotational position of the patient support surface.

7. The method of claim 6, wherein the sensor is arranged to measure the absolute rotational position of the patient support surface with respect to the direction of gravity towards earth.

8. The method of claim 6, wherein the sensor includes an inclinometer.

9. The method of claim 1, wherein determining the relationship between each of the encoder readings and the respective positions of the patient support surface comprises determining at least one parameter, the at least one parameter being representative of a characteristic of the radiotherapy patient positioning apparatus.

10. The method of claim 9, wherein the at least one parameter comprises a plurality of parameters, each parameter being representative of a respective characteristic of the radiotherapy patient positioning apparatus.

11. The method of claim 10, wherein the method further comprises;
taking one or more readings from the sensor and encoder at a plurality of positions, wherein the number of positions of the plurality of positions is equivalent to at least the number of parameters of the plurality of parameters.

12. The method of claim 9, wherein the at least one parameter is representative of a characteristic of the movement mechanism.

13. The method of claim 9, wherein the characteristic is at least one of: movement mechanism backlash or encoder offset.

14. A system comprising:
a radiotherapy apparatus, the radiotherapy apparatus comprising:
a patient support surface;
a sensor configured to measure a position of the patient support surface;
a movement mechanism configured to move the patient support surface;
an encoder configured to encode an internal position of the movement mechanism;
a processor; and
memory, with instructions stored thereon which, when performed by the processor, cause the processor to:
take one or more readings from the sensor and the encoder at a first position of the patient support surface;
cause the movement mechanism to move the patient support surface to a second position;
take one or more readings from the sensor and the encoder at the second position; and
analyze the one or more readings from the sensor and encoder at the first position and the one or more readings from the sensor and encoder at the second position to determine a relationship between each of the encoder readings and the respective positions of the patient support surface.

15. A non-transitory computer-readable medium containing instructions that, when executed by a processor, cause a radiotherapy system to;
take one or more readings from a sensor and an encoder of the radiotherapy system at a first position of a patient support surface of the radiotherapy system;
cause a movement mechanism of the radiotherapy system to move the patient support surface to a second position;
take one or more readings from the sensor and the encoder at the second position; and
analyze the one or more readings from the sensor and encoder at the first position and the one or more readings from the sensor and encoder at the second position to determine a relationship between each of the encoder readings and the respective positions of the patient support surface.

16. The system of claim 14, wherein a smallest change of the internal position that the encoder is able to resolve causes a positional change in the patient support surface that is smaller than the smallest change in the patient support surface that the sensor is able to resolve.

17. The system of claim 14, wherein the instructions further cause the processor to:
cause the movement mechanism to move the patient support surface to a third position;
make a first estimate of the third position based at least in part on at least one of the one or more readings from the sensor;
use the determined relationship to make a second estimate of the third position, wherein the second estimate is based at least in part on at least one of the one or more readings from the encoder; and
compare the first estimate and the second estimate of the third position.

18. The system of claim 17, wherein comparing the first estimate and the second estimate includes determining whether the first estimate is within a predefined tolerance of the second estimate.

19. The non-transitory computer-readable medium of claim 15, wherein a smallest change of an internal position of the movement mechanism that the encoder is able to resolve causes a positional change in the patient support surface that is smaller than the smallest change in the patient support surface that the sensor is able to resolve.

20. The non-transitory computer-readable medium of claim 15, wherein the instructions further cause the radiotherapy system to:
cause the movement mechanism to move the patient support surface to a third position;
make a first estimate of the third position based at least in part on at least one of the one or more readings from the sensor;
use the determined relationship to make a second estimate of the third position, wherein the second estimate is based at least in part on at least one of the one or more readings from the encoder; and
compare the first estimate and the second estimate of the third position, wherein comparing the first estimate and the second estimate includes determining whether the first estimate is within a predefined tolerance of the second estimate.
